(19) United States
(12) Patent Application Publication
(10) Pub. No.: US 2025/0025688 A1
(43) Pub. Date: Jan. 23, 2025

(54) REVERSIBLE ELECTROPORATION SYSTEM COMPRISING MIDDLE CONNECTOR

(71) Applicant: MMILR INC., Daejeon (KR)

(72) Inventors: Jin Hee MOON, Sejong-si (KR); Mi So LEE, Sejong-si (KR); Myoung Hwa HONG, Yongin-si (KR); Soon Jung OH, Yongin-si (KR)

(73) Assignee: MMILR INC., Daejeon (KR)

(21) Appl. No.: 18/713,305

(22) PCT Filed: Nov. 24, 2021
(86) PCT No.: PCT/KR2021/017431

§ 371 Date: Oct. 8, 2024

(30) Foreign Application Priority Data
Oct. 18, 2021 (KR) 10-2021-0138684

(51) Int. Cl. A61N 1/32; A61N 1/04

(52) U.S. Cl. CPC A61N 1/327; A61N 1/0412

(57) ABSTRACT

Proposed is a reversible electroporation system including a middle connector combined with a cradle, the system using an electroporation needle including an internal drug channel and an electrode pattern formed on the external surface thereof, so as to allow the application of electric pulses and the formation of electric fields at a lesion site of a patient and enable a corresponding drug to be delivered, thereby enabling an accurate and stable procedure for a diseased part of the body of the patient, wherein the middle connector has a seating part so as to be stably fixed on the body without slipping, and has a first mesh-type fixing part placed therein so that, when the needle is inserted into a certain part of the body, an electroporator can be inserted at an accurate position together with the mesh-type fixing part, and when more stable fixation is required according to the convenience and need of an operator, an electroporator cradle including a second mesh-type fixing part is further provided so that a quick and safe procedure is performed.

### TECHNICAL FIELD

The present disclosure relates to a reversible electroporation system and, more particularly, to a reversible electroporation system including a middle connector combined with a cradle, the system using electroporation needle including an internal drug channel and an electrode pattern formed on the external surface thereof, so as to allow the application of electric pulses and the formation of electric fields at a lesion site of a patient and enable a corresponding drug to be delivered, thereby enabling an accurate and stable procedure for a diseased part of the body of the patient.

### BACKGROUND ART

Electroporation is a technique that creates nanometer-sized pores on the surface of a cell membrane by applying electric pulses of nanosecond to millisecond duration to cells at a certain range of intensity using electrical stimulation. Since its discovery in the 1970s, electroporation has been applied to various fields of biotechnology for decades. Micropores on the cell membrane surface created under appropriate electric pulse conditions have a reversible property of disappearing after a certain period of time, and this type of electroporation is called reversible electroporation. Reversible electroporation is mainly used to introduce substances such as hydrophilic drugs, genes, enzymes, and antibodies into cells.

On the other hand, micropores created by strong electric pulses do not disappear and act as a mechanism that ultimately causes the cells to lose their vitality, and this type of electroporation is called irreversible electroporation. Although irreversible electroporation not a desirable phenomenon in terms of reversible electroporation, technological approaches that take advantage of irreversible electroporation are also being made.

Electroporation has been utilized for a long time as a method to kill microorganisms in the food industry or as a method for in vitro gene therapy, but with the development of technology, electroporation has received great attention as a medical technology for treating cancer, and much research is being conducted thereon. Applications of electroporation encompass the delivery of DNA, RNA, siRNA, peptides, proteins, antibodies, drugs or other substances to a variety of cells such as mammalian cells, plant cells, yeast, other processing cells, bacteria, other microorganisms and cells from the human body. Electrical stimulation during the delivery process may also be used for cell fusion in the production of other fused cells.

In in vitro cell fusion, cells are suspended in a buffer or medium favorable for cell survival, and the cell suspension is placed in a rectangular cuvette into which one or more flat electrodes for electrical discharge is inserted. That is, when a mixed solution containing the cells and foreign molecules, including nucleic acids, drugs, and other compounds to be injected into the cells, is placed between the electrodes and an electric pulse is applied from the outside, small pores are formed on the surface of the initial cell membrane, allowing some surrounding foreign molecules to move into the cells, and when the external power source is turned off, the pores on the cell membrane surface disappear and the cell membrane returns to its initial state. Using this phenomenon, substances that cannot pass through the cell membrane may be injected into cells.

As another example, in the case of clinical electroporation applied directly to the human body (in vivo), a needle containing at least one electrode is directly inserted into or touches an affected area to induce an electrical pulse into cells, and when pores are temporarily created in the cell membrane of cancer cells, etc., a drug is injected through the pores.

In other words, by combining a common method of administering vaccines and other pharmaceutical agents to body tissues, in which a vaccine or an agent is injected into muscle or skin tissue using a syringe and a needle, with a method of applying electroporation pulses of electrical energy by separately constructing an electrode capable of applying a pulse or combining an electrode with a needle, drugs may be delivered directly to cells in the tissue.

Typically, an electroporator consists of: an output unit that outputs an electrical signal or pulse-type electrical signal; a monitoring unit that monitors the electrical signal output from the output unit; an alarm unit that generates an alarm signal when a failure is detected in the monitoring unit and generates a treatment completion signal when treatment is completed; a storage unit that stores patient data or treatment data; a display unit that displays a patient data input screen or treatment screen; an input unit that receives patient data or treatment setting data; and a control unit that sets the electrical signal according to the treatment setting data input from the input unit and controls the output unit to apply the electrical signal, wherein the output pulse is supplied to a target area using a separate handpiece-type electrode applicator or a fusion-type applicator integrated into a needle for injecting a drug.

In this regard, according to patent document, Korean Patent No. 10-0756252 (registered Aug. 31, 2007 title of the invention: METHOD AND APPARATUS FOR USING ELECTROPORATION MEDIATED DELIVERY OF DRUGS AND GENES), the disclosed technology relates to a method and an apparatus for in vivo electroporation therapy, wherein using electroporation therapy (EPT) as described in the invention, tumors are treated by a combination of electroporation using the apparatus of the invention, and chemotherapeutic agents cause regression of tumors in vivo. In one embodiment, the invention provides an EPT method utilizing low voltage, long pulse lengths for inducing cell death. The embodiment includes a system for clinical electroporation that includes a needle array electrode having a “keying” element that determines the set point of the therapy voltage pulse, and/or selective array switching patterns.

The above patent document discloses a pulse generator that includes an EPT treatment instrument and an electrode applicator and is capable of programming and controlling the pulse duration, voltage level, and output for the electrode applicator, wherein the generated pulse is transmitted into the tissue through at least one electrode needle of the electrode applicator. In addition, the applicator is configured such that not only can electrodes be connected to the applicator, but also a medicine supply unit can be connected to a tube. However, the above patent document only discloses the basic structure of the reversible electroporation treatment, but does not disclose a supporting method or characteristic configuration to ensure that, when performing reversible electroporation on the human body, an electrode applicator is accurately positioned on the relevant tissue area in a situation where needles that cause skin trauma need to penetrate the skin barrier, or to allow an operator to perform the procedure without restrictions on movement in a typical treatment environment, such as where drug injection tubes or power lines are complicatedly intertwined.

According to another patent document, Japanese Patent No. 6860497 (registered Mar. 30, 2021 title of the invention: SYSTEMS AND METHODS FOR IMPROVED TISSUE-SENSING BASED ELECTROPORATION), an adaptive control method for controlling EP pulse parameters during electroporation (EP) of cells or tissue using an EP system includes: providing a system for adaptive control to optimize EP pulse parameters including EP pulse parameters; applying voltage and current excitation signals to the cells; obtaining data from the current and voltage measurements, and processing the data to separate the desirable data from the undesirable data; extracting relevant features from the desirable data; applying at least a portion of the relevant features to a trained diagnostic model; and estimating EP pulsing parameters on the basis of an outcome of the applied relevant features to optimize the EP pulsing parameters such as pulse width, pulse number, amplitude/field strength, and frequency.

Although the above patent document discloses a control method for programming pulse conditions and controlling pulse operation characteristics and output characteristics based on feedback in an EP system involving data logging, and main components such as a pulse generator A, an applicator C, and a foot pedal, no specific solution or characteristic configuration is presented regarding a middle connector that includes a drug pumping means and an ampoule (drug container) and also has a holding function, configuration of a cradle to insert the applicator into the desired body part at the correct angle and position, and a convenience device that maximizes the ease of treatment.

### DOCUMENT OF RELATED ART

### Patent Document

(Patent Document 1) Korean Patent No. 10-0756252 (registered Aug. 31, 2007 title of the invention: METHOD AND APPARATUS FOR USING ELECTROPORATION MEDIATED DELIVERY OF DRUGS AND GENES)
(Patent Document 2) Japanese Patent No. 6860497 (registered Mar. 30, 2021 title of the invention: SYSTEMS AND METHODS FOR IMPROVED TISSUE-SENSING BASED ELECTROPORATION)

### DISCLOSURE

### Technical Problem

The present disclosure is intended to solve the above problems occurring in the related art. An objective of the present disclosure is to provide a reversible electroporation system that allows accurate and stable application of pulses and injection of drugs to a lesion site inside the body, and maximizes the convenience of the procedure without restricting the operator's movement or activities, so as to improve the shortcomings of a conventional electroporation system. In the case of a conventional electroporation system, a pulse generator and an applicator are connected to a long power line and drug injection tube, which can cause restrictions on activities in the treatment environment. Moreover, it is difficult to miniaturize a pump for drug injection and there are limitations in finely controlling the injected drug or preventing waste.

In addition, although during electroporation, it is necessary to minimize unnecessary damage to the skin or areas other than target tissue and to administer an appropriate amount of drug, and an electrode and a needle need to be controlled to be properly inserted into the skin to a lesion inside the body and to the treatment depth, previously, no solutions were provided to effectively solve problems other than the operator's experience. Therefore, an objective of the present disclosure is to provide a reversible electroporation system including a middle connector combined with a cradle to secure a stable and accurate treatment position while maximizing operator convenience.

### Technical Solution

In order to achieve the above mentioned objectives, there is provided a reversible electroporation system including a middle connector, the system including: a pulse generator 300 configured to create an electric field to a target lesion by means electrode; at least one electroporator 200 configured to simultaneously supply a pulse and inject a drug into a target site; and a middle connector 100 configured to mount the electroporator 200 at a correct treatment position and adjust an insertion angle and an insertion depth of the electroporator 200 so that a procedure is performed stably.

That is, the reversible electroporation system including a middle connector according to the present disclosure may include: a pulse generator that applies an electric field to a lesion in a certain part of the body by means of an electrode; an electroporator (applicator) that applies a generated pulse to relevant lesion cells and allows a drug to be injected when a pore is created in a cell membrane; and a middle connector combined with a cradle, which sits on the certain part of the body and stably fixes the electroporator.

In addition, the middle connector may include an ampoule mounting part 120 to which an ampoule is fastened and a pump mounting part 130 to which a pump is fastened, and control pump pressure and drug injection amount.

In addition, the pump mounting part may be connected with the electroporator, a power cable 190, and a drug tube 180, and it is desirable that the pump mounting part be rotatable at various angles to increase operator's freedom of treatment.

In addition, the pulse generator may generate pulses with an intensity ranging from 300 V/cm 2 to 1,500 V/cm 2 and a width of 1 us to 200 μs, and transmit the pulses to the electroporator by means of the middle connector.

In addition, a needle 210 of the electroporator may include: an internal hollow part 220 that allows drug injection for reversible treatment; and an electrode pattern part 250 wound in parallel or spirally along an external surface of the needle.

In addition, the middle connector may include: a seating part 140 that has a streamlined shape at a portion in contact with a surface of a body and is made of an anti-slip means to prevent slipping, so that the middle connector is seated at a specific location on the body without shaking, wherein the seating part may include a belt fixing part 600 that may be fixed to a chair or a treatment bed using a belt to minimize shaking or change in position of the middle connector due to external factors during the procedure.

In addition, the anti-slip means may be made of rubber or may consist of a plurality of airbags capable of expansion and contraction to prevent slipping on the body and ensure a stable fixation.

In addition, inside a main body housing 110 of the middle connector, a first mesh-type fixing part 160 may be provided to accurately position the electroporator at the treatment position.

In addition, the first mesh-type fixing part may be made of a conductive material and may have at least one perforated hole, so that an electric field may be applied through an electroporator needle or directly when touching a portion of a body, and a drug may be applied directly to a skin surface.

In addition, on a side of a main body housing of the middle connector, a guide groove 170 may be formed to adjust a position of a separate electroporator cradle 400.

In addition, a separate electroporator cradle 400 may be mounted in a guide groove 170, and the electroporator cradle 400 may include a second mesh-type fixing part 410, an angle adjustment part 420, a position adjustment guide 430, wherein the electroporator may be moved to an appropriate position by the position adjustment guide, the electroporator may be adjusted or fixed to an angle required for the procedure by the angle adjustment part, and the electroporator may be stably fixed using a through hole of the second mesh-type fixing part and a through hole of a first mesh-type fixing part.

In addition, the electroporator cradle may further include a height adjustment part 440 capable of moving up and down to increase convenience for the procedure depending on a position of an operator or a patient.

In addition, the electroporator cradle may have a shape that protrudes at intervals of 120 degrees in a circumferential direction, and a guide groove 450 may be formed on an inside of the protruded shape so that the electroporator may move along the guide groove, and it is preferable that a fastening groove 460 may be formed at an end of the guide groove to secure the at least one electroporator depending on an extent of the lesion.

In addition, the middle connector may consist of a circular middle connector 500 including a first housing 510, a second housing 520, and a seating pad 530, wherein by using a third mesh-type fixing part 540 provided on an inside of the first housing, and a fourth mesh-type fixing part 550 provided on an inside of the second housing 520, a position of the electroporator may be stably adjusted, and the insertion angle and the insertion depth may also be adjusted.

In this case, the inner central part of the middle connector includes a mesh-type fixing part that may fix the electroporator (applicator), and a separate electroporator cradle that may fix the upper part of the electroporator is mounted on a guide groove provided on one inner side of the middle connector. Thus, there is no need for an operator to continuously hold the electroporator during a procedure, and the electroporator is stably fixed to at least one mesh-type fixing part, so that an accurate treatment position may be secured and a lesion inside the body may be accurately reached.

In addition, since the middle connector includes a pump mounting part where a pump that supplies a drug by pneumatic pressure is seated, and an ampoule mounting part where an ampoule is fastened, a drug is supplied over a short distance to the electroporator placed on the middle connector, making it possible to finely control the drug injection amount without wasting drugs.

In addition, unlike in the conventional case where multiple connection lines are complicatedly intertwined because a pneumatic pump and a drug container exist separately, a drug is supplied from the middle connector placed on the patient's body, there are no restrictions on the operator's movement and activities, preventing sudden accidents and shortening the procedure time.

### Advantageous Effects

According to a reversible electroporation system including a middle connector of the present disclosure,

first, it is possible to insert an electroporator at a precise angle and certain depth into a lesion inside the body.

Second, stable fixation is possible without shaking or changing position during manipulation while the procedure is taking place.

Third, by integrating a pump for drug injection and a drug ampoule in the middle connector, the drug delivery distance between electroporators is shortened, preventing waste of drugs and making it possible to finely control the drug injection amount.

Fourth, by integrating a pump, a drug ampoule, and a fixing part, the connection line is simplified, which reduces restrictions on the operator's movement and allows for quick and accurate treatment from the operator's perspective.

### DESCRIPTION OF DRAWINGS

FIG. 1 schematically shows a reversible electroporation system according to the present disclosure;

FIG. 2 schematically shows a cross-sectional view of a middle connector of the reversible electroporation system according to the present disclosure;

FIG. 3 schematically shows a top view of the middle connector of the reversible electroporation system according to the present disclosure;

FIG. 4 shows an electroporator cradle mounted on the middle connector according to an embodiment of the present disclosure;

FIG. 5 schematically shows a pump mounting part provided on one side of the middle connector of the present disclosure;

FIG. 6 schematically shows the electroporator cradle of the present disclosure fastened to a guide groove and the detailed configuration of the cradle;

FIG. 7 shows the height adjustment of the electroporator cradle according to an embodiment of the present disclosure;

FIG. 8 schematically shows another type of electroporator cradle according to an embodiment of the present disclosure;

FIG. 9 schematically shows a circular type middle connector according to an embodiment of the present disclosure;

FIG. 10 schematically shows the configuration of the electroporator according to the present disclosure; and

FIG. 11 schematically shows the configuration of an electroporator electrode needle according to an embodiment of the present disclosure.

### BEST MODE

The detailed description of the present disclosure described below refers to the accompanying drawings, which show by way of example specific embodiments in which the present disclosure may be practiced. The embodiments are described in sufficient detail to enable those skilled in the art to practice the present disclosure. It should be understood that the various embodiments of the present disclosure are different from one another but are not necessarily mutually exclusive. For example, specific shapes, structures and characteristics described herein with respect to one embodiment may be implemented in other embodiments without departing from the spirit and scope of the present disclosure. In addition, it should be understood that the location or arrangement of individual components within each disclosed embodiment may be changed without departing from the spirit and scope of the present disclosure. Therefore, the detailed description below is not intended to be taken in a limiting sense, and the scope of the present disclosure is limited solely by the appended claims, together with all equivalents claimed therein, if properly described. Similar reference numerals in the drawings refer to identical or similar functions across various aspects.

Hereinafter, in order to enable those skilled in the art to easily practice the present disclosure, preferred embodiments of the present disclosure will be described in detail with reference to the attached drawings.

### (Reversible Electroporation System Including Middle Connector)

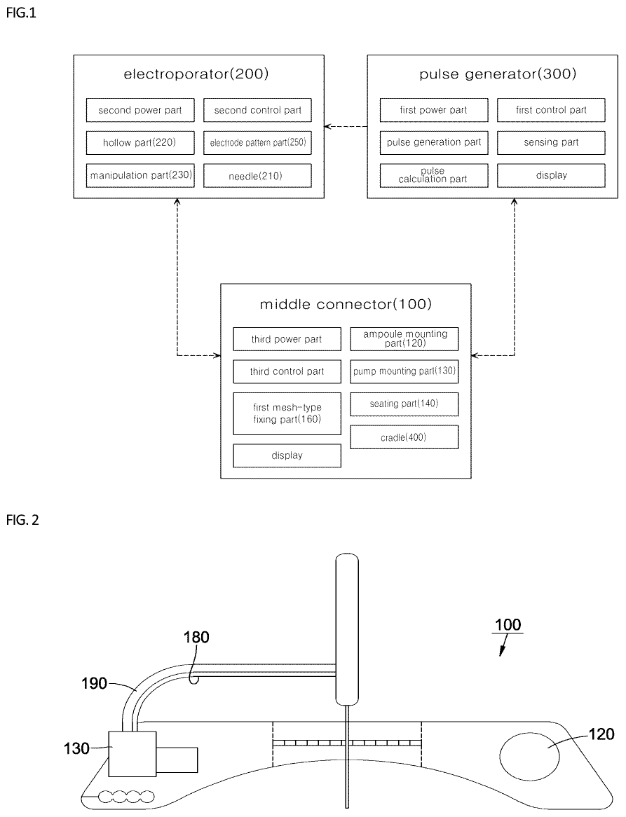

FIG. 1 schematically shows a reversible electroporation system according to the present disclosure. A reversible electroporation system including a middle connector according to the present disclosure may be a reversible electroporation system or an irreversible electroporation system depending on pulse control and drug injection.

However, it is preferable that a reversible electroporation system according to an embodiment of the present disclosure is a reversible electroporation system because the system according to an embodiment of the present disclosure uses an electroporator including a needle having an internal drug channel and an electrode pattern formed on the external surface thereof to apply electric fields to human skin or a lesion site inside the body. That is, the system is a device that can be used in a treatment method in which a drug is injected when pores are formed in a cell membrane, and the cell membrane is restored after the application of the electric fields is stopped and a certain period of time has elapsed. In addition, since there is no need to generate excessive electrical stimulation, shock to the body or cell damage may be minimized.

As shown in FIG. 1, the reversible electroporation system according to the present disclosure includes a middle connector 100, an electroporator 200, a pulse generator 300.

In this case, the pulse generator 300 may supply and control power to operate the middle connector and the electroporator by means of a first power part and a first control part. In particular, pulses applied to a certain part of the body by means of the middle connector and electroporator may be controlled by sensing, by a sensing part, an electrical signal that comes from a location where the corresponding pulses are applied and by optimizing, by a pulse calculation part, pulse parameters such as pulse width, pulse number, amplitude/electric field strength, and frequency.

In addition, the electroporator 200 includes a second power part and a second control part, a hollow part 220 as a drug channel, an electrode pattern part 250, a manipulation part 230, a needle 210.

One or more needles 210 may be provided, and inside each needle, a drug channel 220 such as a hollow part is formed so that a drug delivered from the middle connector may be injected into the skin or inside the body. In addition, the electrode pattern part 250 is formed on the external surface of each needle 210, so that a pulse generated by the pulse generator 300 is transmitted to the electroporator through the middle connector, and an electric field is created at a target lesion site through the electrode pattern part 250, thereby forming pores in the cell membrane of the lesion cell, and the drug is injected into the pores to initiate treatment.

In this case, the electrode pattern part 250 may be formed parallel to the external surface of the needle 210 or may be wound in a spiral shape along the external surface.

Conventionally, an electrode is constructed separately from a needle that is used to inject a drug. In the present disclosure, however, an electrode is integrated into a needle, and a patterned metal electrode has relatively excellent electrical conductivity, electric field effect, and efficiency depending on their characteristics and may be configured in various forms.

For reference, the pulse generator 300 may be configured separately or integrated into the middle connector.

The middle connector 100 includes a third power part and a third control part, an ampoule mounting part 120, a pump mounting part 130, a seating part 140, a first mesh-type fixing part 160, a cradle 400, and a display.

In this case, the ampoule mounting part 120 is not configured in the pulse generator or separately as in the conventional case, but is integrated into the middle connector combined with a cradle for an electroporator, which makes it easy to fasten an ampoule, and the ampoule mounting part 120 is connected to the electroporator with a drug tube 180 to deliver the drug. Since the ampoule is integrated into the middle connector, a drug delivery section is much shorter than that of a typical electroporation system, and thus it can be helpful not only for quantitative drug injection or fine control, but also for simplifying the configuration and preventing drug waste.

As in the case of the ampoule mounting part, the pump mounting part 130 is not configured in the pulse generator to provide pneumatic pressure from the pulse generator, but is configured such that a pump may be coupled to the middle connector 100. Thus, in proportion to the shortened distance to the electroporator, a pump of relatively small size and capacity may be used, which helps reduce the size and cost of the overall product and is also effective in fine pressure control.

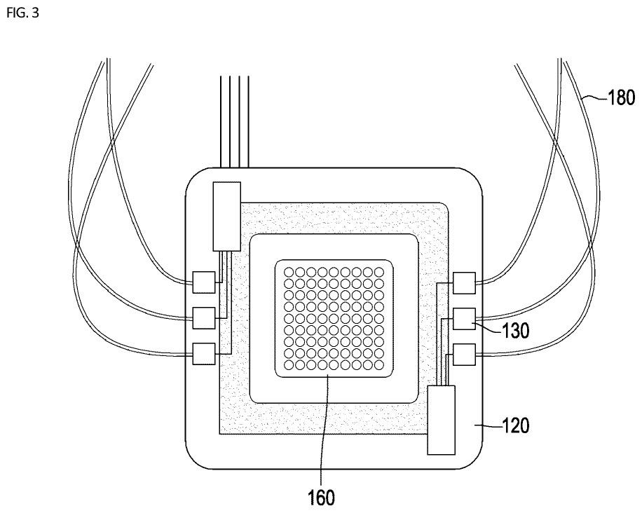

FIG. 2 schematically shows a cross-sectional view of a middle connector of the reversible electroporation system according to the present disclosure, and FIG. 3 schematically shows a top view of the middle connector of the reversible electroporation system according to the present disclosure.

As shown in FIGS. 2 and 3, the middle connector according to the present disclosure includes a power part to which a power cable 190 is connected, the ampoule mounting part 120 to which an ampoule is fastened, a pump mounting part 130 to which a pump is fastened, the first mesh-type fixing part 160, and the drug tube.

The power cable allows the electroporator to operate by means of the middle connector due to the power and pulse provided from the pulse generator. The ampoule mounting part 120 to which the ampoule is fastened is connected to the pump mounting part 130, and the pump mounting part 130 is connected to the electroporator 200, so that according to the control signal, the pump (not shown) transfers the drug from the ampoule to the electroporator due to pressure.

The first mesh-type fixing part 160 is provided inside the middle connector and has a plurality of mesh type holes for fixing the electroporator, so that it is possible to stably maintain a certain angle and direction of the electroporator due to the fixing force that holds a needle in the inserted position and the fixing force by the first mesh-type fixing part when the needle is inserted into a target area of the body

In treating the skin rather than treating the inside of the body, that is, depending on the treatment target or location, an additional electroporator cradle 400 may be used to ensure more stable fixation.

Furthermore, the first mesh-type fixing part 160 may be made of a conductive material such as metal and may apply a pulse and create an electric field by directly contacting the target range for which electroporation treatment for the skin is required, such as in the case of skin cancer. In addition to a hole for fixing the electroporator, at least one injection hole through which a drug is applied may also be configured.

One or more electroporators 200 may be mounted on the first mesh-type fixing part 160 to expand the electric field range depending on the type and scope of the disease.

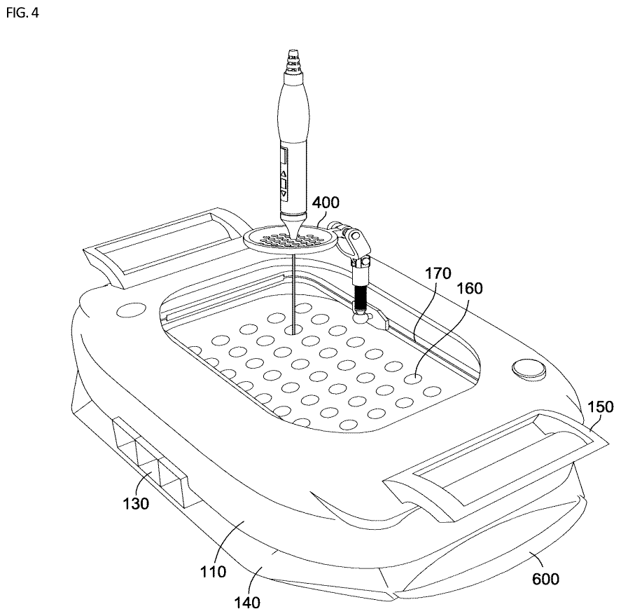

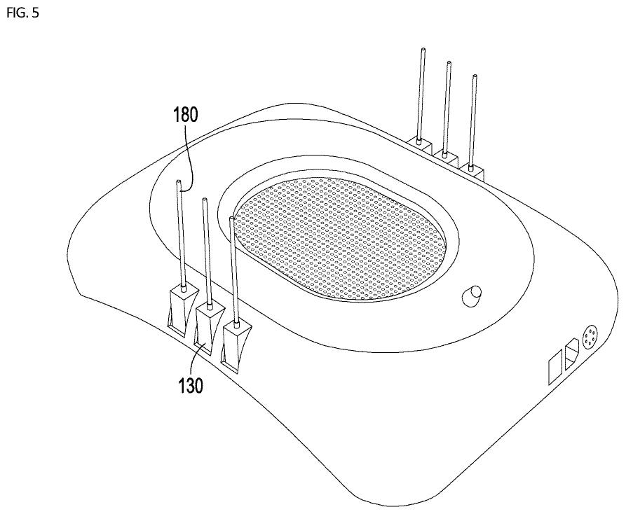

FIG. 4 shows an electroporator cradle mounted on the middle connector and the configuration of a belt fixing part according to an embodiment of the present disclosure.

Referring to FIG. 4, the middle connector may be used by attaching the electroporator cradle 400 to a guide groove 170 formed on the inside of the middle connector depending on the treatment target, location, and operator's needs. The middle connector may be moved along the guide groove depending on the location of the target body part or the operator's convenience, and may be integrated into the middle connector if necessary.

In addition, when more stable fixation is required, a belt is wrapped around the patient's body, chair, surgical bed, operating table, etc. (not shown) by using the belt fixing part 600, so that the middle connector may be firmly seated and fixed to prevent it from moving unnecessarily or slipping off in certain situations.

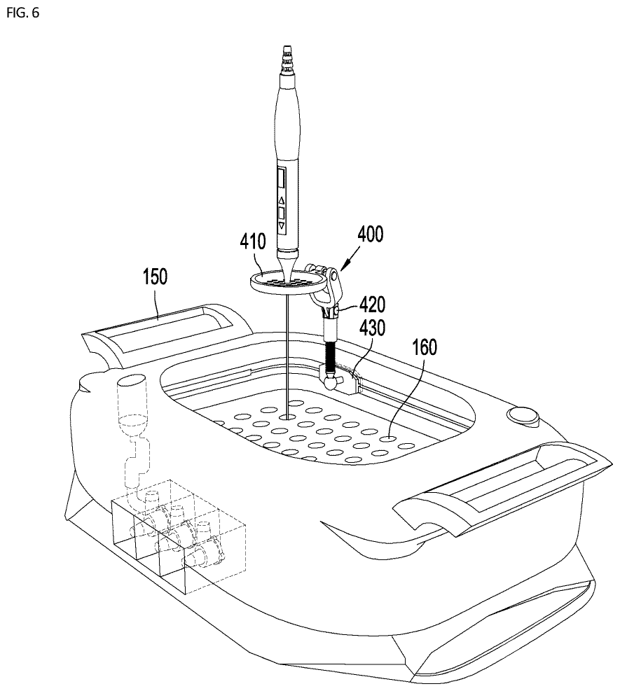

FIG. 5 schematically shows a pump mounting part provided on one side of the middle connector of the present disclosure.

Referring to FIG. 5, the middle connector is equipped with the pump mounting part 130. At this time, the pump mounting part may be configured to rotate at various angles such that the drug tube is not fixed and moves together when the electroporator 200 is moved so that the operator's activities are not restricted during the procedure, and also to increase the operator's degree of freedom during the procedure, depending on the operator's needs. The pump mounting part may be implemented as a typical device configuration that may be freely rotated and fixed within 90 degrees without being limited to a certain angle.

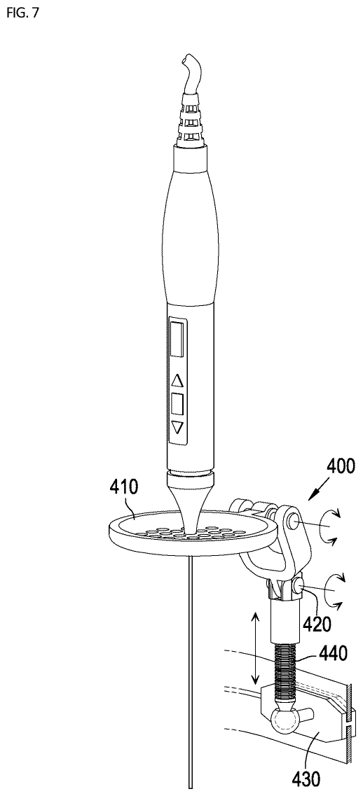

FIG. 6 schematically shows the electroporator cradle of the present disclosure fastened to a guide groove and the detailed configuration of the cradle, and FIG. 7 shows the height adjustment of the electroporator cradle according to an embodiment of the present disclosure.

Referring to FIGS. 6 and 7, the electroporator cradle 400 includes a second mesh-type fixing part 410, an angle adjustment part 420, a position adjustment guide 430, and a height adjustment part 440.

The second mesh-type fixing part 410, together with the first mesh-type fixing part 160 located in the middle connector, allows the electroporator 200 to be properly fixed according to the stable operating position, and insertion angle, direction, and depth of the electroporator 200, and may be flexibly adjusted to fit the fixing portion of the electroporator 200 like the first mesh-type fixing part 160.

The angle adjustment part 420 and the height adjustment part 440 are also used to adjust the second mesh-type fixing part so that the needle may be inserted or contacted at the appropriate position, angle, and depth on the target body part.

In addition, the position adjustment guide 430 is fastened to the guide groove 170 of the middle connector, and the position of the position adjustment guide 430 may be adjusted according to the convenience of the operator.

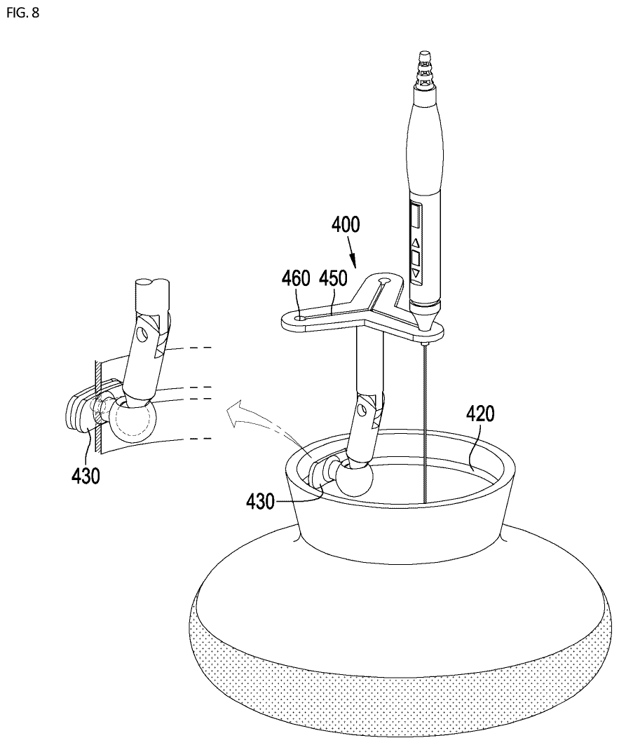

FIG. 8 schematically shows another type of electroporator cradle according to an embodiment of the present disclosure.

Referring to FIG. 8, the electroporator cradle 400 does not have the shape of the mesh-type fixing part, but has a protruding shape in three directions including a guide groove 450 and a fastening groove 460. Each protruding part has an interval of 120 degrees in the circumferential direction, and the electroporator 200 may be moved along the guide groove 450, and at least one electroporator 200 may be fixed by the fastening groove 460, thereby targeting at least one body part or providing a pulsed electric field to a wide range of parts.

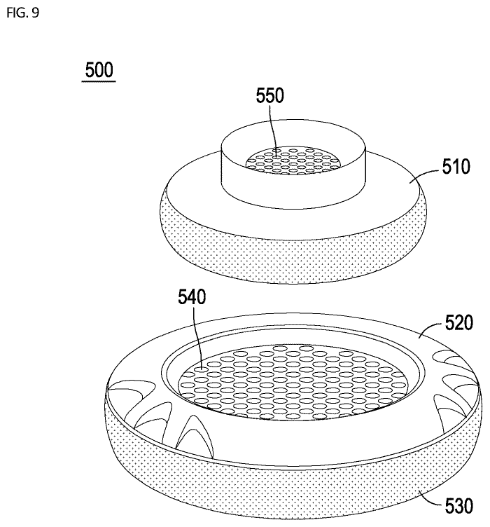

FIG. 9 schematically shows a circular type middle connector according to an embodiment of the present disclosure.

Referring to FIG. 9, the circular type middle connector 500 includes an upper first housing 510, a lower second housing 520, a seating pad 530, a third mesh-type fixing part 540, a fourth mesh-type fixing part 550.

The middle connector 500 may be implemented in various shapes, and in the embodiment of the present disclosure, a circular shape is disclosed.

The first housing 510 includes the third mesh-type fixing part 550 on the inside thereof to fix the electroporator, the function of which is the same as the first and second mesh-type fixing parts. In addition, the seating pad 530 that is in contact with the body is provided at the lower part of the first housing 510, and may perform the same function as the seating part 140 of the middle connector. Like the seating part 140 of the middle connector, the seating pad 530 is made of rubber to prevent slipping on the body and ensure a stable procedure, or is made of a plurality of airbags capable of expansion and contraction or an equivalent configuration. Since the body part targeted for treatment may have various shapes depending on the patient, it is important that the seating part 140 and the seating pad 530 be made of a material that prevents slipping, or that expansion and contraction are controlled to suit the shape of the body to maintain stable contact.

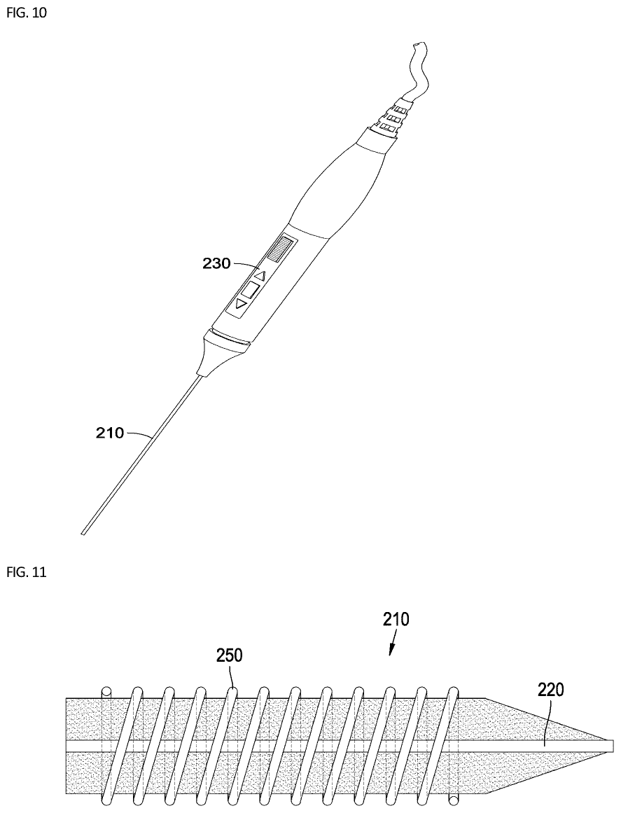

FIG. 10 schematically shows the configuration of the electroporator according to the present disclosure, and FIG. 11 schematically shows the configuration of an electroporator electrode needle according to an embodiment of the present disclosure.

Referring to FIGS. 10 and 11, the electroporator 200 includes a needle 210 and a manipulation part 230 within a main body housing (not shown).

The needle 210 is equipped with an internal hollow part 220 that delivers the drug supplied by the pump from the ampoule (not shown) of the ampoule mounting part 120 to the target body part, and has the electrode pattern part 250 formed surface thereof that transmits the pulse on the external generated by the pulse generator 300 to create an electric field.

At this time, the electrode pattern part 250 may be configured to be parallel or spirally wound along the external surface of the needle. By forming the integrated electrode pattern on the needle 210 in this way, the electric field effect may be maximized.

The embodiments according to the present disclosure described above may be implemented in the form of program instructions that can be executed through various computer components and recorded on a computer-readable recording medium. The computer-readable recording medium may include program instructions, data files, data structures, etc., singly or in combination. Program instructions recorded on the computer-readable recording medium may be specially designed and configured for the present disclosure, or may be known and usable by those skilled in the computer software field.

Examples of computer-readable recording media include magnetic media such as hard disks, floppy disks and magnetic tapes, optical recording media such as CD-ROMs and DVDs, magneto-optical media, such as a floptical disk, and hardware devices specially configured to store and perform program instructions, such as ROM, RAM, flash memory, etc. Examples of program instructions include not only machine language code such as that produced by a compiler, but also high-level language code that can be executed by a computer using an interpreter, etc. The hardware device may be configured to operate as one or more software modules to perform processing according to the present disclosure and vice versa.

In the above, the present disclosure has been described with specific details such as specific components and limited embodiments and drawings, but this is only provided to facilitate a more general understanding of the present disclosure, and the present disclosure is not limited to the above embodiments. A person skilled in the art to which the present disclosure pertains may make various modifications and changes based on this description.

Therefore, the spirit of the present disclosure should not be limited to the embodiments described above, and not only the claims described below, but also all modifications equivalent to the claims will be considered to fall within the scope of the present disclosure.

### DESCRIPTION OF NUMERALS

100: middle connector
200: electroporator
300: pulse generator
110: main body housing
120: ampoule mounting part
130: pump mounting part
140: seating part
150: handle
160: first mesh-type fixing part
170: guide groove
180: drug tube
190: power cable
200: electroporator
210: needle
220: internal hollow part
230: manipulation part
240: connector
250: electrode pattern part
300: pulse generator
400: electroporator cradle
410: second mesh-type fixing part
420: angle adjustment part
430: position adjustment guide
440: height adjustment part
450: guide groove
460: fastening groove
500: circular middle connector
510: first housing
520: second housing
530: seating pad
540: third mesh-type fixing part
550: fourth mesh-type fixing part
600: belt fixing part

1. A reversible electroporation system including a middle connector, the system comprising:
a pulse generator (300) configured to create an electric field to a target lesion by means of an electrode;
at least one electroporator (200) configured to simultaneously supply a pulse and inject a drug into a target site; and
a middle connector (100) configured to mount the electroporator (200) at a correct treatment position and adjust an insertion angle and an insertion depth of the electroporator (200) so that a procedure is performed stably.

2. The system of claim 1, wherein the middle connector comprises an ampoule mounting part (120) to which an ampoule is fastened and a pump mounting part (130) to which a pump is fastened, and controls pump pressure and drug injection amount.

3. The system of claim 2, wherein the pump mounting part is connected with the electroporator, a power cable (190), and a drug tube (180), and is configured to be rotated at various angles to increase operator's freedom of treatment.

4. The system of claim 1, wherein the pulse generator generates pulses with an intensity ranging from 300 V/cm 2 to 1,500 V/cm 2 and a width of 1 us to 200 μs, and transmits the pulses to the electroporator by means of the middle connector.

5. The system of claim 1, wherein a needle (210) of the electroporator comprises:
an internal hollow part (220) that allows drug injection for reversible treatment; and
an electrode pattern part (250) wound in parallel or spirally along an external surface of the needle.

6. The system of claim 1, wherein the middle connector comprises:
a seating part (140) that has a streamlined shape at a portion in contact with a surface of a body and is made of an anti-slip means to prevent slipping, so that the middle connector is seated at a specific location on the body without shaking,
wherein the seating part comprises a belt fixing part (600) that may be fixed to a chair or a treatment bed using a belt to minimize shaking or change in position of the middle connector due to external factors during the procedure.

7. The system of claim 6, wherein the anti-slip means is made of rubber or consists of a plurality of airbags capable of expansion and contraction to prevent slipping on the body and ensure a stable fixation.

8. The system of claim 1, wherein inside a main body housing (110) of the middle connector, a first mesh-type fixing part (160) is provided to accurately position the electroporator at the treatment position.

9. The system of claim 8, wherein the first mesh-type fixing part is made of a conductive material and has at least one perforated hole, so that an electric field may be applied through an electroporator needle or directly when touching a portion of a body, and a drug may be applied directly to a skin surface.

10. The system of claim 1, wherein on a side of a main body housing of the middle connector, a guide groove (170) is formed to adjust a position of a separate electroporator cradle (400).

11. The system of claim 1, wherein a separate electroporator cradle (400) is mounted in a guide groove (170), and the electroporator cradle (400) includes a second mesh-type fixing part (410), an angle adjustment part (420), a position adjustment guide (430),
wherein the electroporator is moved to an appropriate position by the position adjustment guide, the electroporator is adjusted or fixed to an angle required for the procedure by the angle adjustment part, and the electroporator is stably fixed using a through hole of the second mesh-type fixing part and a through hole of a first mesh-type fixing part.

12. The system of claim 11, wherein the electroporator cradle further comprises a height adjustment part (440) capable of moving up and down to increase convenience for the procedure depending on a position of an operator or a patient.

13. The system of claim 11, wherein the electroporator cradle has a shape that protrudes at intervals of 120 degrees in a circumferential direction, and a guide groove (450) is formed on an inside of the protruded shape, so that the electroporator may move along the guide groove, and a fastening groove (460) is formed at an end of the guide groove to secure the at least one electroporator depending on an extent of the lesion.

14. The system of claim 1, wherein the middle connector consists of a circular middle connector (500) including a first housing (510), a second housing (520), and a seating pad (530),
wherein by using a third mesh-type fixing part (540) provided on an inside of the first housing, and a fourth mesh-type fixing part (550) provided on an inside of the second housing (520), a position of the electroporator may be stably adjusted, and the insertion angle and the insertion depth may also be adjusted.
